(19) United States
(12) Patent Application Publication
(10) Pub. No.: US 2022/0054796 A1
(43) Pub. Date: Feb. 24, 2022

(54) Sensory Engagement Toy

(71) Applicant: Trude McGreevy, North Granby, CT (US)

(72) Inventor: Trude McGreevy, North Granby, CT (US)

(21) Appl. No.: 17/399,254

(22) Filed: Aug. 11, 2021

Related U.S. Application Data
(60) Provisional application No. 63/067,482, filed on Aug. 19, 2020.

(51) Int. Cl. A61M 21/02; A63H 3/00; A63H 3/02; A63H 3/28; A63H 9/00; A63H 3/16

(52) U.S. Cl. CPC A61M 21/02; A63H 3/003; A63H 3/02; A61M 2021/0022; A63H 9/00; A63H 3/16; A63H 3/28

(57) ABSTRACT

The present invention relates to a sensory engagement toy for soothing and relaxing individuals, such as children, with sensory overload disorders. More specifically, the sensory engagement toy features a central body portion and a plurality of appendages with different textures extending from the central body. The textures help to calm an individual with autism or other sensory overload disorders. Individuals with sensory overload issues can grasp, pull, throw, and perform other actions with the toy to help calm and relax the individual. The toy has a fabric cover covering the central body portion and the appendages and have internal filling material.

### CROSS-REFERENCE TO RELATED APPLICATION

The present application claims priority to, and the benefit of, U.S. Provisional Application No. 63/067,482, which was filed on Aug. 19, 2020 and is incorporated herein by reference in its entirety.

### FIELD OF THE INVENTION

The present invention relates generally to the field of learning and engagement toys and educational aids. More specifically, the present invention relates to a sensory knitted engagement toy for toddlers and other small children suffering from autism and other sensory-related disorders in order to provide comfort and keep the child focused on the item. The sensory knitted or woven toy of the present invention may be crocheted, woven, sewn or otherwise manufactured made from a soft material and having a plurality of tactile areas for the child to touch. In one embodiment, the toy is a generally round, knitted object with a face and several appendages extending generally outwardly from a central platform. The toy is designed to help soothe and relax children with sensory overload or similar disorders. The invention also includes several different shapes, sizes, colors and faces for the toy or aid which can be used with any child, and enables children to explore, throw, pull and perform other actions with the toy to help maintain composure and refocus him or her, rather than the child becoming upset or throwing a tantrum. The toy or aid can be created with a variety of different accessories which can be tailored to meet the particular characteristics or symptoms of the child. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, many children suffer from autism and other sensory related disorders which can cause problems with communication and social interaction. Autism refers to conditions characterized by challenges with social skills, repetitive behaviors, speech and nonverbal communication, and sensory related disorders which are characterized by persistent challenges with neurological processing of sensory stimuli that interfere with a person's ability to participate in normal activities associated with everyday life. Children suffering with autism and sensory related disorders can get easily agitated, leading to loss of focus, temper tantrums, anxiety, and other similar conditions. These behaviors can create awkward social interactions for both parents and their children. Children with autism and/or sensory related disorders and their parents may have difficulty in combating such disorders and their symptoms and finding effective ways for the parents, guardians and medical assistants to keep the child positively interacting with others.

People including children suffering from autism or sensory related disorders may be advised to undergo therapies and other treatments to feel better or alleviate the symptoms of these disorders. Many treatment therapies such as behavioral therapy, occupational therapy, physical therapy, speech therapy and more are known in the state of the art to help in calming down people suffering from sensory-related disorders. However, the treatment and therapies are costly, time consuming and may disrupt daily activities in order to attend scheduled visits. Not everyone can afford to go for such expensive solutions and people may not have the time available to commit to such tasks. Additionally, medication based treatment is also known to manage autism, however, research has shown that some of these treatments have side effects that can be dangerous to the individuals receiving the treatment. Also, the therapy and medication treatment-based solutions take a long time to be effective, causing inconvenience to children with autism and their parents. Without an effective way to calm down and refocus the attention of the individual suffering from the condition, the child can get overwhelmed and may “shut down” and discontinue activities related to development, education and social interaction.

Therefore, there exists a long-felt need in the art for an engagement and education aid that is specially designed for soothing and calming down individuals suffering from autism and other sensory related disorders. There is also a long-felt need in the art for a toy or other aid that helps in relaxing a child with sensory overload disorders in case the child begins to get upset, stressed and anxious. Additionally, there is a long-felt need in the art for a device that activates proprioceptive senses to calm, focus, and soothe children with sensory overload disorders. Moreover, there is a long-felt need in the art for an alternate solution for treating and combating the symptoms of autism and sensory related disorders. Further, there is a long-felt need in the art for a cost-effective solution that is affordable, easy to use and adapt, can be used to treat the symptoms of autism and sensory-related disorders and can address momentary issues of stress and anxiety. Furthermore, there is a long-felt need in the art for a toy or other aid that assists with refocusing the child, effectively preventing anxiety and temper tantrums in children suffering with autism or other sensory related disorders. Finally, there is a long-felt need in the art for a device or aid that helps quickly in calming down children with autism, and does not create any unintended side-effects.

The subject matter disclosed and claimed herein, in one embodiment thereof, comprises a sensory toy or engagement aid for calming a child with autism or other sensory overload disorders. The sensory toy is designed to be visually stimulating to a child and fashioned after various animals, cartoons, shapes and fanciful and/or imaginary characters. The sensory toy further comprises a central geometrically-shaped body having an outer surface and, depending on the shape, may have a front surface and a rear surface. The exterior or front surface has a face-shaped design and a plurality of flexile appendages or elements that extending generally outwardly from the body. Each appendage has a textured or tactile distal end to appeal to different senses when touched and further appeals to the proprioceptive senses to assist in refocusing and calm the child playing with the toy. The toy is covered with a fabric body and has internal filling material.

In this manner, the novel sensory knitted toy of the present invention accomplishes all of the forgoing objectives, and provides a relatively safe, efficient and cost-effective solution to assist in soothing and relaxing children suffering from autism and/or sensory-related disorders. The sensory knitted toy of the present invention is also user-friendly, as it activates the proprioceptive senses to calm, focus, and soothe children with sensory-related disorders.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sensory knitted toy for calming a child with autism or other sensory overload disorders. The toy or engagement aid may be made by knitting, crocheting, sewing, weaving or other methods of manufacture or combinations thereof. The sensory toy or aid is designed to be visually stimulating to a child and fashioned after various animals, cartoons, shapes and fanciful and/or imaginary characters. The sensory knitted toy further includes a central circular body having an exterior or front surface and a rear surface. The exterior or front surface has a face-shaped design and a plurality of flexile appendages, extensions or elements extending generally outwardly from the circular body. Each appendage has a textured or tactile distal end to appeal to different senses when touched, and further appeals to the proprioceptive senses to assist in refocusing and calm the child playing with the toy. The toy is covered with a soft and washable material such as fabric and has an internal filling material which is compressible.

In a further embodiment of the present invention, a sensory toy or engagement aid to help soothe and relax children with sensory overload disorders is disclosed. The sensory toy includes a body cover made from a soft material such as fabric and an internal compressible filling material. The toy or engagement aid is generally geometrically-shaped, such as a circular central body and a plurality of appendages, elements, or extensions extending outwardly from the central body, the central body has an exterior or front surface and a rear surface, and the front surface has a face design. Each appendage or extension is constructed with various colors and textures, appealing to the proprioceptive senses and assisting with refocusing the child, effectively preventing anxiety and temper tantrums. The appendages or extensions as well as their tactile ends may be changed as the child develops or discovers favorite.

In a further embodiment of the present invention, the internal filling material can be one or a combination of materials from polyurethane, polyester pellets, microbeads, down feathers, and natural shredded rubber that are generally compressible and non-allergenic. The fabric body cover can be selected from natural and synthetic materials such as satin, silk, thick yarn, thin yarn, soft cotton, wool, velvet, polyester and combinations thereof. The fabric body and internal filling materials may also be made from reclaimed, renewable or recycled materials.

In yet a further embodiment of the present invention, a kit comprising a plurality of sensory toys is presented in which the kit has sensory toys, attachment or elements of different designs and shapes which are configured to soothe, engage and relax children with sensory overload disorders. Each sensory toy of the kit has appendages extending from a central body and can be designed with a discrete head, ears, arms, legs, eyes, antennae or other element or extensions. The elements or appendages may be combined or be interchangeable with others to create a variety of reconfigurable elements to provide new adaptations to retain the interest of the child and keep the toy or education aid fresh. The tactile or texture items may be connected by mechanical fasteners such as snaps, hook and loop fasteners or the like. Each appendage has textures or tactile features to appeal to the proprioceptive senses of a child for soothing and refocusing the child. The toys or elements can be grasped, pulled or thrown using the appendages to provide calm to the child.

The sensory toys of the present invention feature a soft and squeezable body member with flexible and extended appendages. The toys are artistically designed to be visually stimulating to a child. The toys can be used by children of any age and enable children to explore, throw, pull, and perform other actions with the toy to help maintain composure and refocus, rather than becoming upset or throwing a tantrum.

The present invention also includes a method for creating a sensory toy or engagement aid. The method includes the steps of initially collecting information on the type of sensory disorder suffered by the child. Next, the size and shape of the toy or engagement aid is determined based on the criteria of the child. A central body element is then designed based on the criteria. Then extensions are selected, as well as one or more tactile elements for connection to the extensions. The extensions are attached to the central body. A further step may include changing the extensions based on developmental behaviors and goals of the child.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a sensory engagement toy of the present invention in accordance with the disclosed architecture;

FIG. 2 illustrates a rear perspective view of one potential embodiment of the sensory engagement toy of the present invention in accordance with the disclosed architecture;

FIG. 3 illustrates a close up perspective view of the textured or tactile surface at a distal end of an appendage of one potential embodiment of the sensory engagement toy of the present invention in accordance with the disclosed architecture;

FIG. 4 illustrates a perspective view of a child playing with one potential embodiment of the sensory engagement toy of the present invention in accordance with the disclosed architecture;

FIG. 5 illustrates a perspective view of one potential embodiment of a plurality of sensory engagement toys of the present invention in accordance with the disclosed architecture;

FIG. 6 illustrates a block diagram of one potential method of making a sensory engagement toy of the present invention in accordance with the disclosed architecture; and

FIG. 7 illustrates a diagrammatic view of one potential embodiment of a kit that can be used to create the sensory engagement toy of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device specially designed for soothing and calming down individuals suffering from autism and other sensory related disorders. There is also a long-felt need in the art for a toy or engagement device that helps in relaxing and soothing a child with sensory overload disorder in case the child begins to get upset, stressed and anxious. Additionally, there is a long-felt need in the art for a device or aid that activates the proprioceptive senses to calm, focus, and soothe children with sensory overload disorders. Moreover, there is a long-felt need in the art for an alternate solution for treating and combating the symptoms of autism and sensory related disorders. Further, there is a long-felt need in the art for a cost-effective solution that is affordable by most individuals and can be used to overcome the symptoms of autism and sensory-related disorders. Furthermore, there is a long-felt need in the art for a toy that assists with refocusing the child, effectively preventing anxiety and temper tantrums in children suffering with autism or sensory-related disorders. Finally, there is a long-felt need in the art for a device that helps quickly in calming down children with autism, and does not have any side effects as well.

The present invention, in one exemplary embodiment, a sensory engagement aid or toy to help soothe and relax children with sensory overload disorders is disclosed. The sensory engagement toy includes a fabric body cover and an internal filling material. The toy has a shape that in one embodiment may be a generally circular shaped central body having a plurality of appendages extending outwardly from the central body. The central body has an exterior surface which may include a front surface and a rear surface. The exterior or forward facing surface has a face design, and each appendage is constructed with various colors and textures, in order to appeal to the proprioceptive senses of the individual and to assist with refocusing the child, effectively preventing anxiety, stress and temper tantrums.

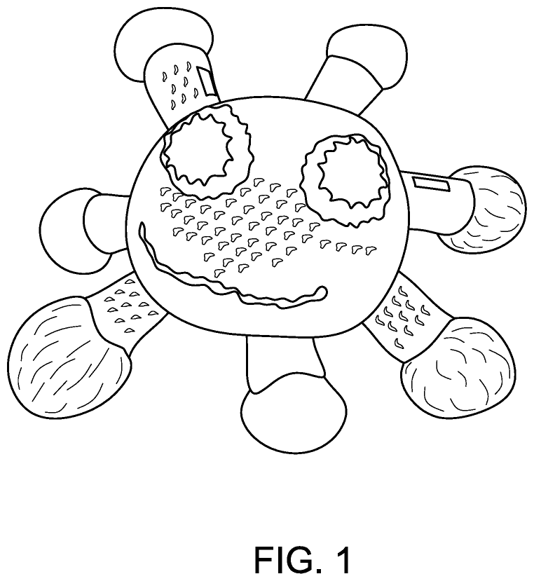

Referring initially to the drawings, FIG. 1 illustrates a front perspective view of one potential embodiment of a sensory engagement toy 100 of the present invention in accordance with the disclosed architecture. More specifically, the sensory engagement toy 100 of the present invention is a portable and relatively lightweight toy for soothing and relaxing an individual, such as a child. In particular, the sensory engagement toy 100 is useful for children and other individuals with sensory overload disorders. The sensory engagement toy 100 can be grasped, pulled, thrown, pressed, hugged or the like by a child for soothing and engagement during periods of stress or anxiety, which may be caused by exposure to changes in environmental stimuli encountered by the child.

The sensory engagement toy 100 of the present embodiment is shaped to accommodate a plurality of appendages laterally extending from the central body. The toy 100 has a fabric covering with soft material internal to the fabric covering. The central-shaped body 102 may include a face-shaped structure having eyes 118, 120 and lips 122 on the exterior or front surface 1020. The appendages extend laterally outward from the central circular body 102 and each appendage has a textured area at the distal end to appeal to the proprioceptive senses of a child playing with the toy 100.

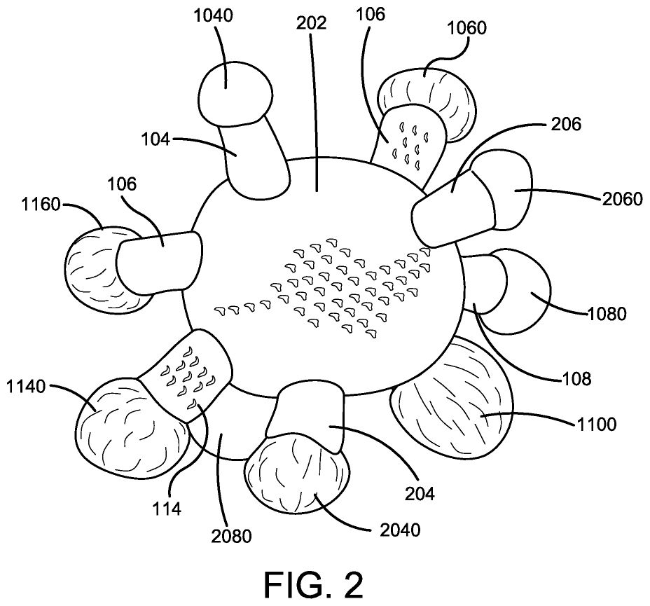

In the present embodiment, the toy 100 has appendages 104, 106, 108, 110, 112, 114, 116 extending laterally from the circular body 102. It should be noted that additional appendages can be extended from the rear surface of the circular central body and are shown in FIG. 2. Each appendage 104, 106, 108, 110, 112, 114, 116 may be knitted, sewn or otherwise attached to the central body 102 and is integrated to the body 102. Further, each appendage 104, 106, 108, 110, 112, 114, 116 can have different textured or tactile surfaces at the distal end or along the sides 105 of the appendage or extension to help with calming a child with autism or other sensory overload disorders.

As best shown in FIG. 1, a first appendage 104 extending from the circular body 102 has a textured or tactile distal end 1040 or side 105, a second appendage 106 extending from the circular body 102 has a textured or tactile distal end 1060, a third appendage 108 extending from the circular body 102 has a textured or tactile distal end 1080, a fourth appendage 110 extending from the circular body 102 has a textured or tactile distal end 1100, a fifth appendage 112 extending from the circular body 102 has a textured or tactile distal end 1120, a sixth appendage 114 extending from the circular body 102 has a textured or tactile distal end 1140, and a seventh appendage 116 extending from the circular body 102 has a textured or tactile distal end 1160. The textured surfaces of the appendages 104, 106, 108, 110, 112, 114, 116 can be different, and the appendages 104, 106, 108, 110, 112, 114, 116 can have different lengths and shapes. The textured surface of the appendages 104, 106, 108, 110, 112, 114, 116 can activate the proprioceptive senses to calm, focus, and soothe children with sensory overload disorders. Each appendage 104, 106, 108, 110, 112, 114, 116 is sufficiently rigid enough to hold upright, while still allowing flexibility and maintaining a soft texture. The appendages 104, 106, 108, 110, 112, 114, 116 may be cylindrical in shape, or may be tapered going towards the distal end. The appendages or elements may have any shape and may also be looped onto the body to create a carrying handle or the like.

The central circular body 102 and the appendages 104, 106, 108, 110, 112, 114, 116 of the toy or engagement element 100 are designed for providing fun and calmness to a child. Actions such as pushing the toy 100 against a surface, pulling the appendages and squeezing or hugging the central body and appendages, activate the proprioceptive senses of a child with sensory overload disorders. The sensory engagement toy 100 is made from a soft and stretchy material that may include materials that are natural, synthetic, recycled, renewable or combinations thereof. In one preferred embodiment, cotton or wool material may be used. The material selected may also or provide the child with a sense of comfort and safety, and well as a feeling of control over their environment and senses.

Proprioceptive input is provided by the engagement toy 100 that is calming for children who are easily overwhelmed by sensory stimulation. The toy 100 regulates the emotional and behavioral responses to sensory stimulation of the children playing with the toy 100. The sensory toy 100 helps children with a sensory-processing disorder and autism by engaging their touch, movement and balance. The toy 100 enhances brain development while helping children playing with the toy 100 to relax, remain calm and overcome fears in a warm and natural environment at home or school.

The engagement toy 100 is available in different shapes, sizes, colors and faces to accommodate any child, and enables children to explore, throw, pull, hug and perform other actions with the toy 100 in order to help maintain composure and refocus. The soft material internal to the fabric covering can be polyurethane foam in the preferred embodiment. Alternatively, the soft material can be cotton or silicon. Any material or combination of materials such as polyester pellets, microbeads, down feathers and natural shredded rubber can also be used for the filling material internal to the fabric covering. The fabric covering can be selected from a group comprised of satin, silk, velvet, thick yarn, thin yarn, soft cotton, wool and velvet. The materials selected should be washable so as to allow for continued and repeated usage.

FIG. 2 illustrates a rear perspective view of one potential embodiment of the sensory engagement toy 100 of the present invention in accordance with the disclosed architecture. More specifically, a plurality of appendages or extensions is extended laterally and outwardly from the rear surface 202 of the engagement toy 100. As shown, additional appendages or extensions 204, 206 extend from the exterior rear surface 202. The appendages 204, 206 are similar in structure to the other appendages 104, 106, 108, 110, 112, 114, 116 shown in FIG. 1. The appendages 204, 206 have distal textured or tactile surfaces 2040, 2060 to provide proprioceptive input for regulating the emotional and behavioral responses of the children playing with toy 100. The textured or tactile surfaces may be provided at the ends of the appendages or extensions or along the sides.

Further, from the rear portion of the knitted toy 100, appendages 104, 106, 108, 114, 116 and textured portions 1040, 1060, 1080, 1100, 2080, 1140, 1160 are visible. It should be appreciated that the number and size of the appendages, as well as the area covered by the textured or tactile portions, can be designed based on the size of the circular body 102 of the toy 100. Each appendage can have a textured portion in different colors and configurations and no two of the textured or tactile portions need to be the same. Alternatively, all of the textured or tactile portions may be the same or nearly the same if such texture is appealing to the child.

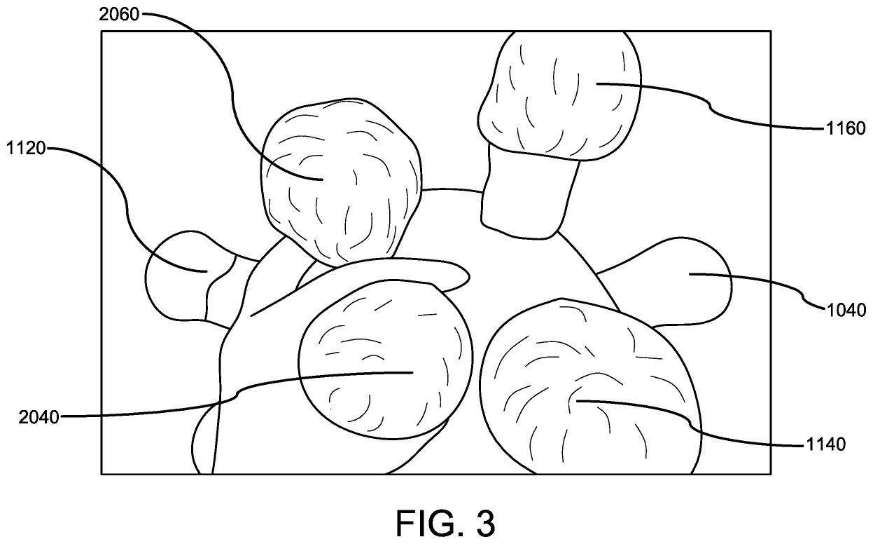

FIG. 3 illustrates a close up perspective view of the textured or tactile surface at a distal end of an appendage of one potential embodiment of the sensory engagement toy 100 of the present invention in accordance with the disclosed architecture. The textured or tactile portion of each appendage provides proprioceptive input for regulating emotional and behavioral responses of a child playing with the toy. More specifically, when one or more textured or tactile portions 1120, 2060, 1160, 1040, 1140, 2040 (including distal or side portions of other appendages present on the toy 100 but not shown in FIG. 3) are squeezed, touched, hugged, or pulled by a child, a calming and soothing effect is provided to the child to help maintain composure and refocus.

Each texture may be the same or different from the other textures/appendages. The appendages stimulate the tactile system through various textures. The appendages also help a child to grasp the toy 100 easily. The materials used for toy 100 are natural, hypoallergenic, non-toxic, BPA, phthalate and PVC free.

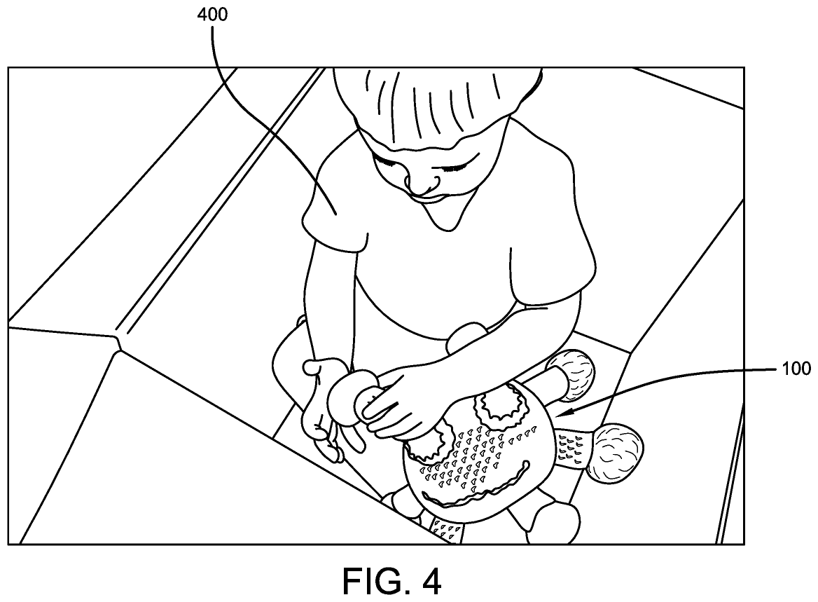

FIG. 4 illustrates a perspective view of a child playing with one potential embodiment of the sensory engagement toy 100 of the present invention in accordance with the disclosed architecture. The sensory engagement toy 100 can be used for creating sensory elements and calming a child 400 with autism or other sensory overload disorders. The child 400 can easily play or otherwise engage with the toy 100 by grasping the toy 100 with one or more appendages. The textures of the appendages appeal to different senses when touched to provide calmness and aid in soothing the child. The child 400 can grasp, pull, throw, hug and perform other actions with the toy 100 for soothing purposes and to attain calmness. The textured or tactile areas on the appendages appeal to the proprioceptive senses and assist with refocusing the child 400, effectively preventing anxiety and temper tantrums.

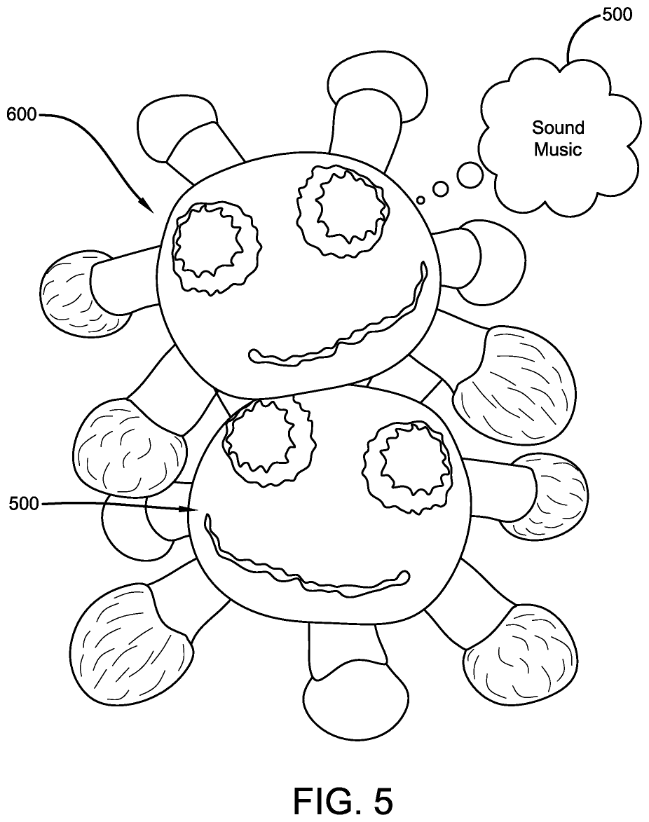

FIG. 5 illustrates a perspective view of one potential embodiment of a plurality of sensory engagement toys 500, 600 of the present invention in accordance with the disclosed architecture. More specifically, the sensory engagement toys 100, 500, 600 of the present invention can be fabricated in multiple designs and shapes and can often be fashioned after various animals, cartoon characters, logos and fanciful and/or imaginary characters. The sensory engagement toy is artistically designed to be visually stimulating to a child while providing calmness to a child. As illustrated, the knitted sensory engagement toys 100, 500, 600 can come in various colors, sizes and designs.

In the preferred embodiment, the sensory engagement toys 100, 500, 600 of the present invention have a face-shaped design on the exterior or front facing surface having facial features such as eyes and mouth. The toys 100, 500, 600 are designed to help soothe and relax children with sensory overload disorders, and to stimulate emotional and sensory growth. In many embodiments, appendages attached to the body may form things such as a discrete head, ears, arms, legs, eyes, antennae, etc. to make the toy more aesthetically appealing and interesting to enhance engagement. Based on the design of the toy, the appendages can have different colors and textures. In one embodiment of the present invention, the engagement toy can have a sound producing unit 510 in an inner area of the body, which upon squeezing the central body produces one or more preconfigured audio sounds or messages to soothe and calm a child playing with the toy.

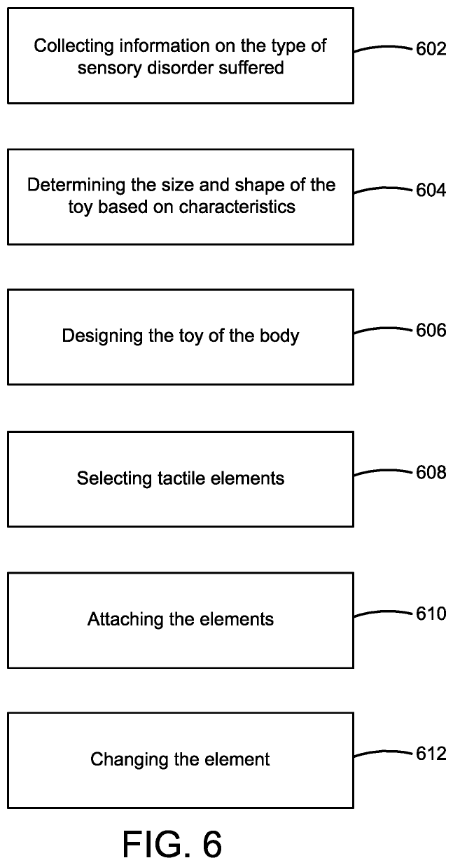

FIG. 6 illustrates a block diagram of one potential method of making a sensory engagement toy 100, 500, 600 of the present invention in accordance with the disclosed architecture. The method includes the steps of initially collecting information on the type of sensory disorder suffered by the child at 602. Next, the size and shape of the toy or engagement aid is determined based on criteria of the child at 604. Then designing a central body element based on the criteria at 606. Then extensions are selected at 608 as well as one or more tactile element for connection to the extensions. The extensions are attached to the central body at 610. A further step may include changing the extensions at step 612 based on developmental behaviors and goals of the child.

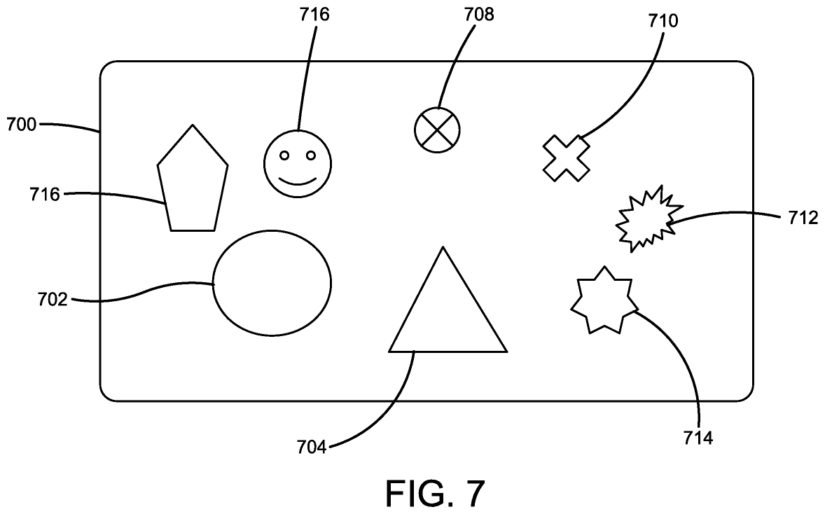

FIG. 7 illustrates a diagrammatic view of one potential embodiment of a kit that can be used to create the sensory engagement toy 100, 500, 600 of the present invention in accordance with the disclosed architecture. More specifically, the kit is provided in a package 700, and comprises a plurality of sensory elements 708, 710, 712, 714 for attachment to appendages or extension elements 716. The kit has sensory toys of different designs and shapes 708, 710, 712, 714 configured to soothe and relax children with sensory overload disorders. Each sensory toy of the kit has appendages extending from a central body element and can be designed as a discrete head, ears, arms, legs, eyes, antennae, etc. A face 706 may be provided for attachment to the central body, which may have different shapes 702, 704 depending on the needs of the child. Each appendage has textures to appeal to the proprioceptive senses of a child for soothing and refocusing the child. The toys can be grasped, pulled or thrown using the appendages to provide calm to the child. The diameter of the central body of the knitted toy can be in the range 6″-12″, for example. The length of the appendages can be in the range 4″-8″, for example.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sensory knitted toy”, “crochet sensory toy”, “knitted toy”, “sensory toy”, and “toy” are interchangeable and refer to the sensory engagement toys 100, 500, 600 of the present invention and it should be understood that the sewing, knitting, crocheting and other manufacturing techniques are interchangeable and various combinations of manufacturing the engagement toy may be used to make the toy.

Notwithstanding the forgoing, the sensory engagement toys 100, 500, 600 of the present invention and their various components can be of any suitable size and configuration as is known in the art without affecting the overall concept of the invention, provided that they accomplish the above-stated objectives. One of ordinary skill in the art will appreciate that the size, configuration, and material of the sensory engagement toys 100, 500, 600 as shown in the FIGS. are for illustrative purposes only, and that many other sizes and shapes of the sensory engagement toys 100, 500, 600 are well within the scope of the present disclosure. Although the dimensions of the sensory engagement toys 100, 500, 600 are important design parameters for user convenience, the sensory engagement toys 100, 500, 600 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sensory engagement toy comprising:
a body having a shape, an exterior surface, a soft covering material and an inner area filled with a compressible internal filling;
a plurality of appendages attached to the body, wherein each of the plurality of appendages extend outwardly from the body; and
a textured or tactile element attached to each of the plurality of appendages.

2. The sensory engagement toy as recited in claim 1, wherein the exterior surface is comprised of a front facing surface and a face.

3. The sensory engagement toy as recited in claim 1 further comprising a sound emitting device in the inner area.

4. The sensory engagement toy as recited in claim 1, wherein the soft covering material is selected from a group consisting of a satin, a silk, a thick yarn, a thin yarn, a soft cotton, a wool, a velvet, a polyester and a combination thereof.

5. The sensory engagement toy as recited in claim 1, wherein the soft covering material is selected from a group consisting of a reclaimed material, a renewable material and a recycled material.

6. The sensory engagement toy as recited in claim 1, wherein the compressible internal filling is selected from a group consisting of a polyurethane, a plurality of polyester pellets, a plurality of microbeads, a plurality of down feathers, and a natural shredded rubber.

7. The sensory engagement toy as recited in claim 1, wherein each of the soft covering material and the compressible filling material are comprised of a non-allergenic material.

8. The sensory engagement toy as recited in claim 1, wherein the textured or tactile element is on at least one of a distal end or side of each of the plurality of appendages.

9. The sensory engagement toy as recited in claim 1, wherein the textured or tactile elements are interchangeable.

10. The sensory engagement toy as recited in claim 9, wherein the textured or tactile elements are interchangeable based on a sensory-related disorder of a user.

11. A method for making a sensory engagement toy, the method comprising the steps of:
collecting information on a sensory-related disorder suffered by a user;
determining a size and shape of the sensory engagement toy based on a criteria of the user;
designing a central body element based on the criteria;
selecting one or more extensions and one or more tactile elements for connection to each of the one or more extensions; and
attaching the one or more extensions to the one or more extensions.

12. The method as recited in claim 11 further comprising a step of changing the one or more extensions based on a set of developmental behaviors and goals of the user after the step of selecting.

13. The method as recited in claim 11, wherein the sensory engagement toy comprises a shaped body having an exterior surface and an internal cavity.

14. The method as recited in claim 13, wherein the exterior surface is comprised of a soft covering material selected from a group consisting of a satin, a silk, a thick yarn, a thin yarn, a soft cotton, a wool, a velvet, a polyester and a combination thereof.

15. The method as recited in claim 13, wherein the internal cavity is filled with a compressible material selected from a group including polyurethane, polyester pellets, microbeads, down feathers, and natural shredded rubber.

16. The method as recited in claim 13, wherein a covering on the exterior surface and a compressible filling material in the internal cavity are comprised of at least one of a reclaimed material, a renewable material and a recycled material.

17. The method as recited in claim 11 further comprising a step of manufacturing the central body element by one of a knitting, a weaving, a sewing, a crocheting or a combination thereof after the step of designing.

18. A kit for creating a sensory engagement toy, the kit comprising:
a package;
at least one body shape;
a plurality of tactile elements;
a plurality of appendages;
a soft outer covering for the at least one body shape; and
a compressible material positioned in an inner cavity of the at least one body shape, wherein the plurality of tactile elements are attached to the plurality of appendages.

19. The kit for creating a sensory toy as recited in claim 18, wherein the plurality of tactile elements may be attached to a side or an end of the plurality of appendages.

20. The kit for creating a sensory toy as recited in claim 18, wherein the plurality of tactile elements are interchangeable.
